(19) United States
(12) Patent Application Publication
(10) Pub. No.: US 2019/0083560 A1
(43) Pub. Date: Mar. 21, 2019

(54) ANTI-INFLAMMATORY EXTRACT

(71) Applicant: NATUREX S.A., Avignon (FR)

(72) Inventors: Alexis LAVAUD, Sorgues (FR); Antoine BILY, Vedene (FR); Marc ROLLER, Morieres Les Avignon (FR); Simona BIRTIC, Cavaillon (FR)

(21) Appl. No.: 16/091,848

(22) PCT Filed: Apr. 6, 2017
(86) PCT No.: PCT/GB2017/050958

§ 371 Date: Oct. 5, 2018

(30) Foreign Application Priority Data
Apr. 6, 2016 (FR) 1653022

(51) Int. Cl. A61K 36/21; B01D 11/02; A23L 33/105; A61K 8/9789; A61P 29/00; A61P 39/06; A61Q 19/08

(52) U.S. Cl. CPC A61K 36/21; B01D 11/0288; A23L 33/105; A61K 8/9789; A61K 2236/333; A61P 39/06; A61Q 19/08; A23V 2002/00; A61K 2236/15; A61P 29/00

(57) ABSTRACT

The invention relates to a plant extract obtained from the aboveground part of a plant of the genus Aerva. The plant extract is advantageously obtained from the variety Aerva javanica (Burm.f.) Juss. ex Schutt, by means of an extraction method comprising a step of contacting said aboveground part with at least one physiologically acceptable extraction solvent. The plant extract of this type is of interest in particular as a component of a composition of the cosmetic, nutraceutical, pharmaceutical or food type.

The present invention relates, generally, to the field of plant extracts. It relates more particularly to plant extracts obtained from or obtainable from plants of the genus Aerva, in particular Aerva javanica (burm.f.) Juss. ex. Schult, processes for providing such an extract, and uses of such extracts. In particular, the present invention relates to an extract or compositions comprising such extracts for use in treating or preventing inflammation.

The listing or discussion of an apparently prior-published document in this specification should not necessarily be taken as an acknowledgement that the document is part of the state of the art or is common general knowledge.

Inflammation is a normal defence phenomenon following an attack on the organism, which has an initiation phase, an amplification phase and a resolution phase. The inflammatory reaction sometimes exceeds its objectives and can be responsible for deleterious effects such as those observed in the case of chronic inflammation.

There are numerous causes of inflammation, such as infectious agents, inert foreign substances, physical agents, post-traumatic cytotissular lesions, etc.

It is possible to distinguish between two types of inflammation, acute inflammation and chronic inflammation. Despite similar actions taking place in the recognition phase and the mechanisms of action involved in the development of the phase of mobilisation of the immune system, these two types of inflammation are very different in their final outcome of resolving or not resolving the inflammation.

Acute inflammation is a natural response to various causes: trauma, physical agents, endogenous or exogenous irritants, or infections.

It evolves in three phases: a vascular phase or initiation phase, a cellular phase or amplification phase, and a resolution phase.

The first phase of the inflammation starts with a “recognition” reaction resulting from the differentiation of the haematopoietic stem cells (HSCs): lymphocytes, monocytes and macrophages or by circulating proteins such as antibodies, complement proteins (which are present in serum and form part of the innate immunity), and Hageman factors involved in blood coagulation.

The recognition phase is preceded by a multitude of chain reactions involving a group of cells and mediators which vary as a function of the intensity and location of the inflammation.

The final phase corresponds to apoptosis of the polynuclear cells, which allows the inflammation to be stopped; this is referred to as resolution of the inflammation. The immune system has scarcely any involvement in acute inflammation.

Accordingly, in the case of acute inflammation, the resolution phase is marked by a return to homeostasis, successful healing and complete restoration of tissue functions.

If the resolution phase does not occur, the inflammatory response can persist and the inflammation become chronic. It then leads to the formation of scars or fibroses or even to a tissue modification because of its chronicity.

For chronic inflammation, the first steps of the inflammatory response are similar to those described for acute inflammation.

However, unlike acute inflammation, chronic inflammation is due to localised inflammatory persistence, which is perpetuated by the presence, or absence, of any pathogen in the organism.

This persistence of inflammation is responsible for many chronic inflammatory diseases which are often described as autoimmune diseases, such as systemic lupus erythematosus, rheumatoid arthritis, Gougerot-Sjögren's disease or Crohn's disease.

Chronic inflammation therefore fosters the degeneration of cells and tissues such as the joints (arthrosis), the vessel walls (arteriosclerosis) and the pancreas (diabetes). In the long term, it causes a lowering of the immune defences and paves the way for diseases having an inflammatory component (cardiovascular diseases, arthritides, asthma, Alzheimer's disease, irritable bowel syndrome, cancers, autoimmune diseases, etc.). It increases the levels of free radicals and therefore oxidation of the body. In particular, chronic inflammation accelerates ageing.

Chronic inflammation is a major risk factor for several other diseases, including heart disease, cancer and type II diabetes. The entire organism therefore suffers from this latent but present inflammatory state.

Chronic inflammation is distinguished by three things: few apparent symptoms, global action at the level of the organism, and latent and progressive action which lasts over time.

Ageing itself fosters inflammation. More biological markers of inflammation are found in elderly subjects. The reason for this is not yet known, but it is characterised by a general slowing down of antioxidative, hormonal and detoxifying functions, the alteration and improper functioning of the mitochondria, releasing more and more free radicals, the accumulation of toxins or pollutants in the body, etc.

Recently, it has been shown that resolution of the inflammatory episode is not a passive mechanism resulting from the mere disappearance of the proinflammatory mediators.

It mobilises the concerted actions of the cyclooxygenases (COX), the lipoxygenases (LOX) or the cytochromes P450 between different cell types. It permits the synthesis of bioactive lipids which are derived from polyunsaturated fatty acids (PUFA) such as arachidonic acid (ARA), eicosapentaenoic acid (EPA) or docosahexaenoic acid (DHA). These new mediators are named lipoxins, resolvins, maresins or protectins.

Their synthesis pathways are widely described in the scientific literature and are presented briefly below:

### a) Synthesis Pathways of the Arachidonic Acid (ARA)-Dependent Bioactive Mediators

ARA is a non-essential fatty acid of the ω6 family, incorporated into the membrane phospholipids of the cell membrane. Its release by phospholipase A2, the activity of which, under the influence of the glucocorticoids, will permit the formation of derivatives which are involved in the process of inflammation.

During inflammation, the enzymes 12- and 15-LOX will permit the production of intermediate products, which are 12-(S)-HETE and 15-(S)-HETE, respectively. 5-LOX has a dual function in the ARA degradation pathway. 5-LOX either degrades ARA directly to 5-(S)-HETE, which is then metabolised by LTA4 hydrolase to leukotriene B4 (LTB4), or it converts 15-(S)-HETE into lipoxin A4 or B4 (LXA4 and LXB4). LXA4 and LXB4 are the first mediators to have been described which belong to the family of the specialised pro-resolving lipid mediators (SPM).

Starting from ARA, under the action of the COXs, prostaglandin E2 (PGE2) or thromboxane B2 (TBX2) will likewise be synthesised. Under the action of 5-LOX, the conversion of ARA also produces an intermediate (5-(S)-HETE) which is converted by LTA4 hydrolase into leukotriene B4 (LTB4).

Inhibiting the metabolic pathways of ARA in order to reduce certain undesirable effects, inflammatory chronicity and non-resolution, of the PGs and LTB4s appears to be an effective pharmacological direction in order better to respond to an inflammation; this is the case with aspirin, which inhibits COX.

### b) Synthesis Pathways of the Docosahexaenoic Acid (DHA)-Dependent Bioactive Mediators

DHA is an essential polyunsaturated fatty acid of the ω3 type, which is either obtained from the degradation of α-linolenic acid (ALA) or supplied directly by food.

Starting from DHA and the action of 12-LOX, 14-HDoHE will be synthesised, which will give rise to products such as maresin (7-Mar1). Again, starting from DHA, the action of 15-LOX will induce the production of an intermediate (17-HDoHE), a precursor of neuroprotectin D1 (PD1) and of the type-D resolvins (RvD1 and RvD2). In the presence of aspirin, epimers of the type-D resolvins may likewise be obtained: these are aspirin-triggered resolvins (AT-RvDs).

### c) Synthesis Pathways of the Eicosapentaenoic Acid (EPA)-Dependent Bioactive Mediators

Like DHA, EPA is a polyunsaturated fatty acid of the ω3 type which is formed from the degradation of ALA but is also present in food.

Starting from EPA, and in the presence of COXs, an intermediate is synthesised: the intermediate is 18-HEPE which, when converted by LOXs, will permit the production of resolvins E1 and E2 (RvE1 and RvE2).

The mechanism of action of these bioactive molecules is described in greater detail below.

Lipoxins A4 or B4 (or their aspirin-dependent epimers), resolvin D1, maresin 1, resolvins E1 and E2 and neuroprotectin D1, taken separately, have autacoid activities since they act as drugs which are synthesised naturally by our organism during an inflammatory response in order to permit its resolution. They thus have activities which promote the resolution of an inflammatory episode by reducing the infiltration of PMN, by reducing the synthesis of inflammatory cytokines such as TNF-a and the mobilisation of the transcription factor NF-KB. RvE1 and RvE2 also have the ability to induce reepithelialisation of the damaged stratum corneum. RvD1 additionally has anti-nociceptive abilities by virtue of the mobilisation of TRPA1, TRPV3 and TRPV4.

The final outcome of an acute inflammation (i.e. whether it is resolved or becomes chronic) is influenced by several factors, such as the intensity and nature of the injury and the location thereof, but also by a potential excessive response of the host. The passage from inflammation to homeostasis (complete resolution) is a programme which is highly regulated at tissue level. The prostaglandins (PGE2 and PGD2) and leukotriene B4 are particularly involved in the initiation and amplification phases of acute inflammation. PGE2 and PGD2 will in fact promote the conversion of LTB4 to lipoxin A4, leading to the production of resolvins D and E and of protectins. Equally, PGE2, PGD2 and LTB4 may also promote the inflammatory process, inducing a response which is unsuitable, for example excessive, which may become chronic; fibrosis may occur due to substantial destruction of the connective tissue, replacement of which will lead to loss of functionality.

In any case, measurement of the resolution molecules represents a model of choice in the validation of molecules having a positive effect on inflammation and more particularly on repair.

The use of synthetic or artificial ingredients within health/pharmaceutical products has become a concern due to the possible negative effects they may have on consumers' health and care.

This has resulted in an increasing public demand for natural alternatives to artificial ingredients in health/pharmaceutical/cosmetic products.

Therefore, it is of interest to provide plant extracts which have an anti-inflammatory indication, and more precisely an indication against chronic inflammation.

Peroxisomes are small organelles similar to mitochondria containing a series of enzymes involved in the metabolism of oxygenated water (catalase, urate-oxidase, D-amino acid oxidase) and enzymes of the p-oxidation of fatty acids.

Peroxisome proliferator-activated receptors (also referred to as PPARs) are members of the superfamily of the nuclear receptors which regulate in particular the synthesis of lipids, glucose and amino acids within the organism.

Recently, PPARs present in the skin and other organs such as the liver, the kidneys, the heart, the muscles, the brain and adipose tissue, for example, have be found to play an important role in regulating cell functions such as cell proliferation and differentiation, the immune response and apoptosis. PPARs have also been found to aid in the resolution of inflammation.

These receptors can be activated by the peroxisome proliferators or they can also be activated by natural fatty acids. They thus stimulate the expression of genes coding for enzymes involved in peroxisomal and mitochondrial (3-oxidation. This regulatory role causes pleiotropic effects within the organism which are important at the level of various pathologies, thus making these markers a preferential target in the search for new therapies.

Various synthetic molecules exist at present that can act upon certain PPAR-α, -γ/σ, -γ receptors. Unfortunately, however, these molecules can result in unwanted side-effects. For example, an increased risk of heart failure and an increase in adipogenesis for drugs of the thiazolidinedione type.

Natural alternatives based on secondary metabolites other than thiazolidinediones must therefore be found.

Therefore, it is also of interest to provide new plant extracts which can act as an activator of peroxisome proliferator-activated receptors (PPARs).

Recent scientific studies have linked nutrition, in particular calorie restriction, to ageing in good health. It may even be that the increase in lifespan is improved by calorie restriction in animal studies.

SIRT1 is a member of the sirtuins SIRT1-7 family, a family of highly conserved deacetylases linked to NAD+which act as cell sensors for detecting the availability of energy and metabolic processes. SIRT1 is expressed in a wide range of tissues and organs and has been detected in the tissues of the liver, the heart of the pancreas, the muscles, the brain, the skin and adipose tissue. SIRT1 is activated by high NAD+levels, a disorder caused by the status of low cellular energy, for example caused by calorie restriction or physical exercise. Activation of SIRT1 leads to deacetylation of the target proteins which are important for apoptosis, the cell cycle, circadian rhythms, mitochondrial function and the active metabolism, including glucose management, lipid metabolism and energy homeostasis, as well as positive effects on cell protection. Several mice models have been used to study the metabolic function of SIRT1. It has been possible to show that overexpression of SIRT1 causes a reduction in adiposity, serum cholesterol and insulin, while displaying an increased resistance to glucose intolerance and insulin resistance induced by metabolic syndrome and obesity.

In addition, the SIRT1 proteins are also involved in numerous biological processes, in particular in DNA transcription and repair and in the processes of apoptosis and cellular senescence. They are key regulators of cell survival. In older fibroblasts, the endogenous expression of SIRT1 decreases progressively. Old cells produce less sirtuin than young cells. Tests have shown that stimulating the expression of the gene sirtuin in human skin cells allows the ageing thereof to be slowed. The maintenance and activation of SIRT1 is therefore key to combating cutaneous signs of ageing and to maintaining the barrier function of the skin. In fact, a recent study shows the leading role of SIRT1 in maintaining the integrity of the barrier function, thus playing a leading role in maintaining skin hydration as well as the role of protecting against external attacks.

Thus, activating SIRT1 predominantly has beneficial effects for maintaining or improving the homeostasis of the organism, the oxidative stress level, insulin resistance and/or lipid metabolism, the energy balance, physical strength, muscle mass, cellular senescence and thus slowing down the process of ageing, as well as prevention associated with chronic diseases, such as chronic inflammation.

Therefore, it is also of interest to provide new plant extracts having an indication as a sirtuin activator.

The present inventors have surprisingly and unexpectedly found that extracts obtained from or obtainable from the aboveground parts of the plant of the genus Aerva (such as the leaves of the plant), in particular Aerva javanica (burm.f.) Juss. ex. Schult, have biological activity, in particular in humans.

For example, extracts obtained from or obtainable from the aboveground parts of the plant of the genus Aerva, in particular Aerva javanica (burm.f.) Juss. ex. Schult have been shown by the present inventors to have anti-inflammatory activity, including activity which increases levels of specialised pro-resolving mediators, such as those derived from arachidonic acid, eicosapentaenoic acid and/or docosahexaenoic acid.

Extracts obtained from the aboveground parts of the plant Aerva, in particular Aerva javanica (burm.f.) Juss. ex. Schult, may therefore have numerous therapeutic and non-therapeutic uses (e.g. cosmetic uses), such as treating or preventing inflammation.

The extracts of the invention may be useful in the treatment of medical conditions.

### Plant Extract

The invention relates to a plant extract obtained from or obtainable from the aboveground part of a plant of the genus Aerva, which may be referred to hereinafter as the “extract of the invention”.

“Plant extract” is understood to mean in particular a product obtained from a solid/liquid extraction, by means of which phytochemical compounds contained in a plant part (solid body) are solubilised by a solvent.

As will be appreciated by the person skilled in the art, as used herein the term “obtainable from” means that the extract may be obtained from a plant or may be isolated from the plant, or may be obtained from an alternative source, for example by chemical synthesis or enzymatic production. Whereas the term “obtained” as used herein, means that the extract is directly derived from the plant source.

The plants of the genus Aerva belong to the family of the Amaranthaceae.

The genus Aerva is found in particular in tropical regions, principally on the African continent (in particular in Madagascar) but also in south-west and south Asia. “Genus Aerva” includes in particular the following varieties: Aerva artemisioides, Aerva congesta, Aerva coriacea Schinz, Aerva humbertii Cavaco, Aerva javanica (Burman f.) A. L. Juss. ex Schultes, Aerva lanata (L.) A. L. Juss. ex Schultes, Aerva leucura Moq., Aerva madagassica Suess., Aerva microphylla Moq., Aerva revoluta Balf.f., Aerva sericea Moq. island puzzle, Aerva sanguinolenta (L.) Blume, Aerva transvaalensis Gand., Aerva triangularifolia Cavaco.

Preferred plant extracts that are the subject of the present invention are those obtained from or are obtainable from the above ground parts of the species Aerva javanica (Burm.f.) Juss. ex Schutt. (synonyms: Aerva tomentosa Forsk., Aerva persica (Burm. f.) Merrill).

This species is an upright herbaceous plant (which can reach a height of 1.6 metres). It is considered to be perennial and suffrutescent, comprising alternate, linear to suborbicular leaves. Its flowers are unisexual, having oblong to oval tepals, grouped in terminal and comose cylindrical spikes (length: 10 cm). It grows on the waste ground, scrubland, rocky ground or at the edges of forests of tropical and subtropical Africa, in the Indian Ocean and in tropical and subtropical Asia at an altitude of up to 1500 metres.

The term “aboveground part” as used herein, refers to any part of an Aerva plant that is not within the soil which it is growing in. For example, the term “aboveground part” would not include the roots of the plant, but may include parts such as the leaves, the stems, the seeds, the flowers and the fruits. These aboveground parts can be taken individually or in combination. Typically, the “aboveground part” as used in the present invention comprises or consists of the leaves of an Aerva plant, in particular an Aerva javanica plant.

The extract of the invention may be an aqueous extract, an alcohol extract (which includes hydro-alcoholic extracts) or an organic extract. In some instances, an aqueous extract of the invention and an alcohol extract of the invention may be combined to form a mixed extract of the invention. The ratio of aqueous extract to alcohol extract in the mixed extract may be from 1:10 to 10:1, such as from 1:5 to 5:1.

The term “aqueous extract” as used herein, refers to the extract of the invention when the extraction from the aboveground parts of the plant (for example the leaves) has been performed using water as the only solvent.

The term “alcohol extract” as used herein, refers to the extract of the invention when the extraction from the aboveground parts of the plant (for example the leaves) has been performed using an alcohol as the solvent. The alcohol solvent may consist of only alcohol (e.g. 100% alcohol), for example 100% ethanol, or may be a mixture of an alcohol and water (i.e. a hydro-alcoholic solvent), for example, a mix of ethanol and water (hydro-ethanolic solvent), for example, from about 1% to about 99% alcohol (e.g. ethanol) in water, for example the ratio of water to alcohol is from 10/90% v/v to 90/10% v/v or 30/70% v/v to 70/30% v/v, such as 50/50% v/v.

In a preferred aspect, the extract of the invention is an alcoholic extract. In particular, a hydro-alcoholic extract, such as a hydro-ethanolic extract.

The term “organic extract” as used herein, refers to the extract of the invention when the extraction from the aboveground parts of the plant (for example the leaves) has been performed using an organic solvent that is not an alcohol. For example, the organic solvent may be selected from the group consisting of acetic acid, acetone, acetonitrile, benzene, 2-butanone, carbon tetrachloride, chlorobenzene, chloroform, cyclohexane, 1,2-dichloroethane, diethylene glycol, diethyl ether, diglyme (diethylene glycol, dimethyl ether), 1,2-dimethoxy-ethane (glyme, DME), dimethyl-formamide (DMF), dimethyl sulfoxide (DMSO), 1,4-dioxane, ethyl acetate, ethylene glycol, glycerin, heptane, hexamethylphosphoramide (HMPA), hexamethylphosphorous, triamide (HMPT), hexane, methyl t-butyl, ether (MTBE), methylene chloride, N-methyl-2-pyrrolidinone (NMP), nitromethane, pentane, petroleum ether (ligroine), pyridine, tetrahydrofuran (THF), toluene, triethyl amine, o-xylene, m-xylene and p-xylene.

The alcoholic extract of the invention (such as a hydro-alcoholic extract, for example a hydro-ethanolic extract) may comprise, consist essentially of or consist of quercetin di-rhamnosyl glucoside, kaempferol coumaroyl rhamnosyl galactoside isomer, isorhamnetin rhamnosyl rutinoside, rutin, kaempferol coumaroyl rhamnosyl galactoside, and/or kaempferol di-coumaroyl rhamnosyl galactoside.

For example, the alcoholic extract (such as a hydro-alcoholic extract) may comprise, consist essentially of or consist of from about 0.1 to about 0.3% by dry weight of the extract quercetin di-rhamnosyl glucoside, from about 0.05 to about 0.1% by dry weight of the extract kaempferol coumaroyl rhamnosyl galactoside isomer, from about 0.04 to about 0.08% by dry weight of the extract isorhamnetin rhamnosyl rutinoside, from about 0.5 to about 0.15% by dry weight of the extract rutin, from about 0.05 to about 0.1% by dry weight of the extract kaempferol coumaroyl rhamnosyl galactoside, and/or from about 0.1 to about 0.3% by dry weight of the extract kaempferol di-coumaroyl rhamnosyl galactoside.

For the avoidance of doubt, preferences, options, particular features and the like indicated for a given aspect, feature or parameter of the invention should, unless the context indicates otherwise, be regarded as having been disclosed in combination with any and all other preferences, options particular features and the like as indicated for the same or other aspects, features and parameters of the invention.

The term “about” as used herein, e.g. when referring to a measurable value (such as an amount or weight of a particular component in the reaction mixture), refers to variations of ±20%, ±10%, ±5%, ±1%, ±0.5%, or, particularly, ±0.1% of the specified amount.

The skilled person will understand that the extract of the invention may be provided in solid form or in liquid form. By solid form, it is included that the extract may be provided as an amorphous solid, or as a crystalline or part-crystalline solid.

### Extraction Method

A plant extract of the invention may be advantageously obtained from or obtainable from the aboveground parts of a plant of the genus Aerva by carrying out a method comprising a step of contacting the aboveground plant part of the genus Aerva with at least one physiologically acceptable extraction solvent, advantageously followed by a step of removing said solvent.

Typically, the extract of the invention may be obtained by extraction and isolation methods as generally described below, or routine modifications thereof.

The extraction method may comprise or consist of the following: a contacting step, a decolouring step (which may be termed a “bleaching step”) and/or a deodorising step.

The decolouring step and deodorising step may be conducted together as a single step or may be conducted separately. If the decolouring step and deodorising step are conducted separately, the decolouring step may be conducted before or after the deodorising step.

For example, the method for providing an extract of the invention may comprise or consist of the following successive steps:

a) a step of preparing/providing the aboveground part of the plant of the genus Aerva,
b) a step of contacting said aboveground part with the extraction solvent,
c) a step of filtering the mixture to obtain a filtrate of interest, optionally subjecting the solid residue of the aboveground part to a further contacting step followed by a filtering step,
d) optionally, a step of decolouring and/or deodorising said filtrate of interest, and
e) optionally, a step of formulating said plant extract, on a solid support or on a liquid support.

A non-limiting example of the method for producing a plant extract is described below and may comprise:

a) a step of preparing/providing the aboveground part of the plant of the genus Aerva, comprising optionally a grinding step and optionally a drying step,
b) a step of contacting the aboveground part with the extraction solvent, in a dynamic or static manner, for from 1 to 8 hours, preferably at ambient temperature, at reflux or at a temperature ranging from 45 to 55° C.,
c) a step of filtering the mixture to obtain a filtrate of interest, optionally subjecting the solid residue of the aboveground part to a further contacting step followed by a filtering step,
d) optionally, a step of decolouring and/or deodorising the filtrate of interest, and
e) a step of formulating the plant extract on a solid support or by formulation on a liquid support.

During the method of the invention, a concentration step can be optionally carried out after step c) and/or after step d) with the aim of reducing the volume of extraction solvent or filtrate to be treated and in order to obtain a concentrated solution or solid. The concentration step may fully remove the extraction solvent or may partially remove the extraction solvent. For example, the solvent may be concentrated until all the solvent has been removed and only solid extract remains. Typically, the solvent (filtrate) is concentrated (for example, by rotary evaporation) to about 30% to about 70% DM (Dry Material, Dried Matter or Dry Matter) such as about 50% DM.

The extract may then be further dried to a % DM of about 90% to about 99%, such as about 97%. Drying processes that may be used include, but are not limited to, atomization, air drying, oven drying, and sun drying. The drying may be done with or without a carrier.

### Preparation Step a)

The plant extract is prepared or provided from the aboveground part of a plant of the genus Aerva. For example, the plant extract of the invention may be obtained from or obtainable from the leaves, the stems, the seeds, the flowers and/or the fruits of the Aerva plant.

The plant of the genus Aerva may, for example, be a plant of the variety Aerva javanica (Burm.f.) Juss. ex Schult.

The aboveground part is preferably dried then ground or ground then dried before being contacted with the extraction solvent. However, the use of the fresh plant is also conceivable.

When used, grinding is adjusted so as to obtain a ground material having a particle size of from 100 μm to 50 mm, with an average particle size of from 0.5 to 5 mm for example 1 mm. Any suitable grinding technique known in the art may be used.

When used, drying is adjusted so as to avoid deterioration of the plant material during storage thereof; preferably, the percentage of residual water is less than 10%. For example, the ground particles may be dried to a % DM (Dry Material, Dried Matter or Dry Matter) of about 90% to about 99%, such as about 97%. Drying processes that may be used include, but are not limited to, atomization, air drying, oven drying, and sun drying.

### Contacting Step b)

The aboveground part (optionally ground and dried) is contacted with the extraction solvent.

The extraction solvent may be advantageously chosen from:
polar solvents, for example, water, subcritical water, ethyl acetate, acetone, alcohols, such as methanol and ethanol,
eutectic solvents, and
supercritical CO₂.

The extraction solvent may also comprise a mixture of at least two of the abovementioned solvents.

The extraction solvent may be an alcoholic solvent or of an aqueous-alcoholic solvent (hydro-alcoholic solvent) comprising a mixture of water and at least one alcohol.

The aqueous-alcoholic solvent may be a mixture of water and alcohol in which the water/alcohol ratio is preferably from 10/90% v/v to 90/10% v/v (volume/volume) and preferably from 30/70% v/v to 70/30% v/v. More particularly, the water/alcohol ratio used may be advantageously approximately 50/50% v/v.

For example, the aqueous-alcoholic solvent may be a mixture of water and ethanol in which the water/ethanol ratio is preferably from 10/90% v/v to 90/10% v/v (volume/volume) and preferably from 30/70% v/v to 70/30% v/v. More particularly, the water/ethanol ratio used may be advantageously approximately 50/50% v/v.

The term “Eutectic solvent” as used herein includes deep eutectic solvents (DES) which comprise a mixture of an organic salt (ammonium or phosphonium), or without the presence of their counter-ion, and a hydrogen bond donor.

Supercritical CO₂ (carbon dioxide) is a fluid subjected to temperature and pressure conditions such that the temperature applied is greater than a critical temperature (Tc), for example ranging from 35° C. to 80° C., and the pressure applied is greater than a critical pressure (Pc), for example greater than 7.4×10⁶ Pa.

The step of contacting the aboveground part of a plant of the genus Aerva with the extraction solvent is based on a solid-liquid extraction technique, which may optionally include or consist of a macerating step.

This contacting or macerating can be dynamic (with stirring) or static.

Plant/solvent contacting advantageously takes place over a period of from 1 to 8 hours, such as from 1.5 to 4 hours, preferably for about 2 hours.

This contacting can take place at ambient temperature, at reflux or at a temperature ranging from 45 to 55° C., such as about 50° C.

“Ambient temperature” means an extraction carried out at a temperature ranging from 15° C. to 35° C., preferably from 20 to 30° C.

Typically, the plant/solvent ratio (weight of plant in grams/volume of solvent in ml) is in a range of from 1 to 5 to 1 to 20 and preferably 1 to 10 (that is to say, 100 g of aboveground parts per 1 litre of solvent).

Any suitable extraction apparatus may be used. For example, the extract of the invention may be extracted using Soxhlet apparatus.

Any un-dissolved plant material may be removed from the solvent, for example, by filtration, and re-contacting with the extraction solvent. This step may be carried out once or may be repeated, for example, repeated 1 to 5 times.

### Filtering Step c)

The extraction product, obtained at the end of step b), is filtered in order to obtain an extraction mass without plant fibres and insoluble components.

This filtration is generally performed by successive filtration operations, typically using filters of decreasing porosity (for example between 1000 μm and 5 μm) in order to ensure that residues of plant fibres are removed. For example, the filtration may be performed by 1 to 10, such as from 1 to 5 successive filtration operations.

At the end of this filtration step c), a retentate and a filtrate are obtained; the filtrate forms a “crude” plant extract of the invention.

As noted above, a second contacting step b) can be carried out on the retentate in order to remove all or more of the phytochemical compounds from the plant material. Steps b) and c) are then carried out again, under the same conditions as those described above.

The two extraction passes are then combined in order to continue the process of producing the extract of the invention until the “crude” plant extract is obtained.

A concentration step as previously described may be carried out after the filtration step c).

Optional Decolourinq and/or Deodorising Step d)

The “crude” plant extract can optionally be decoloured and/or purified and/or deodorised in order to minimise the impact of the extract in its final application. A decoloured and/or purified and/or deodorised plant extract is thus obtained. This step may also be considered to be a purification step.

In order to decolour and/or purify and/or deodorise the extract, the extract is advantageously contacted with an active material chosen, for example, from bleaching earth, activated carbon, activated charcoal or clays such as bentonite. Preferably, the active material is activated charcoal, such as 10% activated charcoal.

More particularly, a ratio is advantageously used which ranges from 1% to 20% weight/weight (and preferably from 5% to 10%) by weight of active material, based on the dry weight of the extract. The percentages by dry weight are expressed on the basis of the weight of the extract not including, or including in trace amounts, extraction solvent.

Contacting with the active material is advantageously maintained for a period ranging from 15 minutes to 2 hours, more particularly for about 1 hour.

This contacting takes place at ambient temperature to about 50° C.

This mixture is then advantageously filtered in order to remove the active material (decolourising/deodorising material).

This decolouring and/or purifying and/or deodorising step can alternatively be carried out by means of ion exchange resins or adsorbent resins.

A concentration step as previously described may be carried out after the decolouring and/or deodorising step d).

### Optional Step of Formulating the Plant Extract e)

The formulating step advantageously comprises or consists of combining the crude plant extract (which may be concentrated and/or bleached and/or deodorised) with a physiologically acceptable support (excipient).

The support may advantageously be a compound of natural origin which is present in nature or the support may be a compound of natural origin that has been modified so that the support structure is different to the natural product from which it is derived and is therefore not found in nature.

The formulating step may, for example, be chosen from, drying on a solid support and, on the other hand, formulation in a liquid support.

Drying allows the plant extract to be concentrated.

Drying can be carried out by means of a suitable drying installation (an atomiser, a vacuum oven or a drum dryer, for example) in the presence of a solid support.

If a solid support is used, the solid support advantageously consists of a polysaccharide, for example maltodextrin, starch or a natural polysaccharide of the acacia gum type.

Typically, maltodextrin may be used in the present invention. Maltodextrins are commonly used excipients or carriers.

Maltodextrins are defined as starch hydrolysis products with dextrose equivalent less than 20. Dextrose equivalent (DE value) is a measure of the reducing power of starch derived oligosaccharides expressed as percentage of D-glucose on dry matter of hydrolysate and is inverse value of average degree of polymerisation (DP) of anhydro glucose units. As products of starch hydrolysis, maltodextrins contain linear amylose and branched amylopectin degradation products, therefore they are considered as D-glucose polymers joined by a-(1,4) and a-(1,6) linkages.

Although maltodextrins are derived from a natural compound (starch), their structure is different from the initial structure of the natural molecule they derive from (starch). This difference is induced by the hydrolysis process. Thus, maltodextrin structure does not occur in nature.

Other possible excipients or carriers include arabic gum, dextrose, and salt.

The plant extract of the invention, or the solid supported extract may then be in the form of a powder.

If a liquid support is used, the liquid support, of varied viscosity, can be a solubiliser, an emulsifier, a surfactant, an emollient or mixtures thereof, in order to improve the formulation of the extract from the invention in view of the application thereof in the desired fields.

For example, the liquid support may consist of a compound derived from glycol (such as glycerol, propylene glycol, butylene glycol, pentylene glycol, propanediol) or an oily support comprising a surfactant.

“Liquid support” also includes:
polyols;
water with or without preservatives;
ethanol as well as any compound of the alkyl-alcohol type, such as fatty alcohols;
eutectic solvents containing an organic acid (such as citric acid, malic acid, ascorbic acid, lactic acid, glycolic acid) and/or a sugar (such as sucrose, fructose, inositol) and/or an amino acid (such as glycine, proline, arginine, betaine, a choline derivative);
alkyl glucosides, alkyl polyglucosides;
monoglyceride and its derivatives.

Other possible excipients or carriers include mono & diglycerides of fatty acids, MPG, Polysorbate 80, vegetable oil, mono & diglycerides of fatty acids, glucose syrup, glycerin, water and alcohol.

Polyols are also called glycols and correspond to organic compounds comprising at least two alcohol functions (—OH group), such as diols with variable substitutions comprising, without implying any limitation, 1,2-propanediol, ethylene glycol, diethylene glycol, propylene glycol, dipropylene glycol, butylene glycol, pentylene glycol, or mixtures thereof; the organic compounds also include triols, such as glycerol or caprylyl glycol, or mixtures thereof.

Monoglycerides include, without implying any limitation, glycerol monolaurate, glycerol monocaprate, glycerol monocaprylate, glycerol monooleate, glycerol monomyristate, glycerol monopalmitate, glycerol monostearate, or mixtures thereof.

Alkyl glucosides and alkyl polyglucosides include, without implying any limitation, decyl glucoside, arachidyl glucoside, butyl glucoside, caprylyl/capryl glucoside, caprylyl glucoside, cetearyl glucoside, coco-glucoside, ethyl glucoside, isostearyl glucoside, heptyl glucoside, lauryl glucoside, myristyl glucoside, hexadecyl glucoside, octadecyl glucoside, octyldodecyl glucoside, undecyl glucoside, or mixtures thereof.

The plant extract of the invention, or the liquid supported plant extract may then be in the form of a liquid.

### Compositions of the Invention

The extract of the invention may also be provided in the form of a composition, advantageously a cosmetic, nutraceutical, pharmaceutical or food composition, comprising an extract of the invention described previously.

Such a composition is intended in particular for use in or on humans.

As used herein the term “cosmetic composition” is understood as being in particular a preparation which is to be applied to the skin, the hair, or the buccal mucosa in order to clean them, to protect them or to keep them in good condition and/or to improve appearance.

As used herein the term “nutraceutical composition” is understood as being in particular the plant extract, made available in medicinal form and conventionally not associated with foods, and having a beneficial or protective physiological effect, for example, against chronic diseases.

By “pharmaceutically acceptable” we mean that the additional components of the composition are generally safe, non-toxic, and neither biologically nor otherwise undesirable. For example, the additional components are generally sterile and pyrogen free. Such components must be “acceptable” in the sense of being compatible with the extract of the invention and not deleterious to the recipients thereof. Thus, “pharmaceutically acceptable excipients” includes any compound(s) used in forming a part of the formulation that is intended to act merely as an excipient, i.e. not intended to have biological activity itself.

Accordingly, the composition according to the invention contains the plant extract according to the invention, advantageously as an active ingredient.

The pharmaceutical, cosmetic, nutraceutical or food compositions may comprise an extract of the invention in a therapeutically effective amount. As used herein, the term “effective amount” is synonymous with “therapeutically effective amount”, “effective dose”, or “therapeutically effective dose” and when used in reference to treating inflammation refers to the minimum dose of the extract of the invention necessary to achieve the desired therapeutic effect and includes a dose sufficient to reduce a symptom associated with inflammation. Effectiveness in treating inflammation can be determined by observing an improvement in an individual based upon one or more clinical symptoms, and/or physiological indicators associated with the condition. An improvement in inflammation also can be indicated by a reduced need for a concurrent therapy.

The appropriate effective amount of the extract or composition of the invention to be administered to an individual for a particular inflammation can be determined by a person of ordinary skill in the art by taking into account factors, including, without limitation, the type of inflammation, the location of the inflammation, the cause of the inflammation, the severity of the inflammation, the degree of relief desired, the duration of relief desired, the particular dosage of extract of the invention that is used, the rate of excretion of the extract of the invention used, the pharmacodynamics of the extract of the invention used, the nature of other compounds that may be included in the composition, the particular formulation, the particular route of administration, the particular characteristics, history and risk factors of the patient, such as, e.g., age, weight, general health and the like, or any combination thereof.

Additionally, where repeated administration of the extract or composition of the invention is used, an effective amount of the extract of the invention will further depend upon factors, including, without limitation, the frequency of administration, the half-life of the extract of the invention, or any combination thereof.

The compositions (e.g. a pharmaceutical, cosmetic, nutraceutical or food composition) of the invention advantageously comprise from 0.001% to 20% by weight of the plant extract, based on the total weight of the final composition (according to whether the extract is dried or formulated on a liquid support), and more particularly from 0.01% to 10%.

The compositions according to the invention may contain at least one other active ingredient, in combination with the plant extract according to the invention.

The compositions according to the invention may further comprise a physiologically acceptable excipient, adapted in particular according to the intended form and the desired route of administration of the composition.

As used herein, references to pharmaceutically acceptable excipients may refer to pharmaceutically acceptable adjuvants, diluents and/or carriers as known to those skilled in the art.

The physiologically acceptable excipient may be a cosmetically or dermatologically acceptable excipient.

Cosmetic, dermatological and/or food acceptable ingredients/excipients include those known in the art (including those also referred to herein as pharmaceutically acceptable excipients) and can be natural or non-natural, i.e. their structure may occur in nature or not. In certain instances, they can originate from natural compounds and be later modified so that it is distinct from the natural product from which it originated (e.g. maltodextrin).

Suitable carriers include, but are not limited to, inert solid diluents or fillers, sterile aqueous solutions and various organic solvents. Examples of solid carriers are lactose, terra alba, sucrose, cyclodextrin, maltodextrin, dextrin, talc, gelatine, agar, pectin, acacia, magnesium stearate, magnesium hydroxide; stearic acid, arabic gum, modified starch and lower alkyl ethers of cellulose, saccharose, silicon dioxide. Examples of liquid carriers are syrup, vegetables oils, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

Examples of other physiologically acceptable carriers that may be used in the present invention include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN (for example, polysorbate based emulsifiers, such as polysorbate 20, 40, 60 or 80), polyethylene glycol (PEG), and PLURONIC (for example, block copolymers based on ethylene oxide and propylene oxide).

The skilled person will understand that extracts of the invention (e.g. in the form of compositions, such as pharmaceutical, cosmetic, nutraceutical or food compositions) may be administered to a patient or subject (e.g. a human or animal patient or subject) by any suitable route, such as by the enteral, topical, oral, rectal, nasal, pulmonary, buccal, sublingual, transdermal, intracisternal, intraperitoneal, and parenteral (including subcutaneous, intramuscular, intrathecal, intravenous and intradermal) route.

For an enteral route, the compositions (more particularly the nutraceutical, food and/or pharmaceutical composition) may be in the form of tablets, gelatin capsules, dragées, syrups, suspensions, solutions, powders, granules, emulsions, microspheres or nanospheres, or lipid or polymeric vesicles permitting controlled release.

For a parenteral route, the compositions may be in the form of solutions or suspensions, for perfusion or for injection.

The compounds, or extract of the invention, used according to the invention, by the enteral or parenteral route, may be administered in a daily dose of approximately from 0.001 mg/kg to 1000 mg/kg of body weight in from 1 to 3 administrations, more particularly from 0.01 mg/kg to 100 mg/kg of body weight in from 1 to 3 administrations.

Typically, the extract of the invention may be administered in an amount of from about 100 mg/day to about 2000 mg/day, or from about 500 mg/day to about 1500 mg/day, or about 1000 mg/day. If the extract is administered in the form of a pharmaceutical or veterinary or food, feed or pet food supplement or food, feed or pet food composition comprising the extract, the extract would be present in an amount to provide the above dosages of extract. For example, the food composition may comprise from about 100 mg to about 2000 mg or from about 500 mg to about 1500 mg, or about 1000 mg/day of the extract of the invention and the pharmaceutical composition may comprise 10 mg, 20 mg, 30 mg, 40 mg, 50 mg, 60 mg, 70 mg, 80 mg, 100 mg, 250 mg, 500 mg, 1000 mg, 1500 mg or 2000 mg/day of the extract of the invention, such that the pharmaceutical composition may be administered one or more times per day in order to provide from about 100 mg to about 2000 mg or from about 500 mg to about 1500 mg, or about 1000 mg of the extract of the invention.

Pharmaceutical, cosmetic, nutraceutical or food compositions of the invention may consist of or consist essentially of the extract of the invention and pharmaceutical or food/nutraceutical composition.

For the avoidance of doubt, in this specification when we use the term “comprising” or “comprises” we mean that the extract or composition being described must contain the listed ingredient(s) but may optionally contain additional ingredients. When we use the term “consisting essentially of” or “consists essentially of” we mean that the extract or composition being described must contain the listed ingredient(s) and may also contain small (for example up to 5% by weight, or up to 1% or 0.1% by weight) of other ingredients provided that any additional ingredients do not affect the essential properties of the extract or composition. When we use the term “consisting of” or “consists of” we mean that the extract or composition being described must contain the listed ingredient(s) only.

Preferably, the compositions (more particularly a cosmetic, dermatological or pharmaceutical composition) may be packaged in a form suitable for application by the topical route.

For a topical route of this type, the compositions according to the invention may be more particularly intended for the treatment of the skin and mucosa.

The compositions according to the invention may be in the form of unguents, creams, milks, ointments, powders, impregnated pads, solutions, gels, sprays, lotions or suspensions. The compositions according to the invention may also be in the form of microspheres or nanospheres or lipid or polymeric vesicles or polymeric patches and hydrogels permitting controlled release. The compositions administered by the topical route may be either in anhydrous form or in aqueous form.

Again generally, the compositions may be, without implying any limitation, in the form of a powder, an emulsion, a microemulsion, a nanoemulsion, a suspension, a solution, a lotion, a cream, a gel cream, an aqueous or ethanolic gel, a serum, an aerosol, a dispersion of lipid vesicles.

“Emulsion” is understood in particular as being an emulsion of the water-in-oil or oil-in-water type or a double emulsion (oil-in-water-in-oil or water-in-oil-in-water).

The cosmetic or pharmaceutical compositions according to the invention may contain a solvent chosen according to the final application and the desired administration. For example, the solvent may be chosen from water, ethanol, glycerol and propylene glycol.

The compositions may also contain at least one additive conventionally used in the cosmetic, pharmaceutical, nutraceutical or food field, chosen from an emollient or humectant, a gelling agent, a surfactant, an anti-agglomerant, an oil, an active agent, a colourant, a pigment, a perfume, a flavouring or a sunscreen.

In general, the compositions may further contain inert or also active additives or combinations of such additives, namely, for example, at least one of the following additives:
one or more emollients, wetting agents or hydrating agents, such as glycerol or other glycols; these additives may be present in the composition in proportions of approximately from 0.1% to 40%, preferably from 1% to 10%, based on the total weight of the composition;
one or more gelling or texturising agents chosen, without this list being exhaustive, for example, from cellulose derivatives, gums of natural origin (guar, acacia, carob, alginate, carrageenan, pectin, tragacanth) or of biotechnological origin (xanthan), clays (laponite, bentonite, montmorillonite, kaolin), acrylate copolymers such as acrylates/C10-30 alkyl acrylate; these additives may be present in the composition in proportions of approximately from 0.1% to 15%, based on the total weight of the composition;
one or more surfactants, of varied polarity and ionicity, which are present in proportions of approximately from 0.1% to 10%, preferably from 0.5% to 8%, based on the total weight of the composition;
one or more volatile or non-volatile, petrochemical-, silicone- or plant-based fatty substances commonly called oils, such as, without implying any limitation, a vegetable oil (jojoba oil, sunflower oil, sesame oil, tea oil, tsubaki oil, argan oil, cranberry oil, shea butter, mango butter), isododecane, octyldodecanol, squalane, dimethicone, preferably in proportions of approximately from 0.1% to 30%, more particularly from 0.5% to 15%, based on the total weight of the composition;
one or more active agents of natural or synthetic origin having biological activity, such as, for example, without implying any limitation, vitamins, hyaluronic acid, depigmenting agents or plant extracts, which can be used in proportions of approximately from 0.001% to 10% and more particularly from 0.1% to 5%, based on the total weight of the composition;
one or more colourants of natural, animal or synthetic origin, in proportions of approximately from 0.01% to 10%, based on the total weight of the composition;
one or more anti-agglomerating agents or compacting agents such as magnesium stearate, silica, maltodextrin or cellulose and the derivatives thereof, in proportions of approximately from 0.1% to 20%, based on the total weight of the composition;
perfumes, antioxidants, preservatives or agents for improving the taste, for example.

### Therapeutic Uses

The extract or compositions of the invention may be useful in the treatment of medical conditions.

It is in particular shown in the present application that the extract or compositions of the invention are suitable for use as:
an agent for preventing and/or treating (which may include curing) inflammation, advantageously an agent for preventing and/or treating (which may include curing) chronic inflammation, yet more advantageously an agent for preventing and/or treating (which may include curing) chronic inflammation associated with skin disorders, and/or
an activating agent of peroxisome proliferator-activated receptors (PPARs), advantageously following an inflammatory stimulation, and/or
an agent for the treatment of skin disorders, in particular skin disorders associated with chronic inflammation, and/or
an activating agent of sirtuins, more particularly of SIRT1, advantageously following an inflammatory stimulation.

Thus, according to the present invention, there is provided an extract or composition of the invention for use as a drug.

The present invention also provides an extract or composition of the invention for use in preventing and/or treating inflammation.

The present invention also provides an extract or composition of the invention for use in preventing and/or treating chronic inflammation.

The present invention further provides an extract or composition of the invention in preventing and/or treating chronic inflammation associated with skin disorders.

The extract or compositions of the invention may activate peroxisome proliferator-activated receptors (PPARs). Thus, the present invention also provides an extract or composition of the invention for use as a peroxisome proliferator-activated receptor (PPAR) agonist. For example, the present invention provides an extract or composition of the invention for use as a peroxisome proliferator-activated receptor (PPAR) agonist following an inflammatory stimulation.

Thus, the present invention also provides a plant extract or composition of the invention as a peroxisome proliferator-activated receptor (PPAR) agonist for use in treating skin disorders and pathologies, and/or controlling oxidative stress at neuronal level, acting within the scope of cerebral traumas, and/or promoting the phenomenon of healing, and/or improving cellular cohesion, and/or managing body weight and associated disorders.

The skin disorders are typically associated with chronic inflammation, and may be selected from atopic dermatitis, seborrhoeic dermatitis, acne, psoriasis, couperose, erythrosis, telangiectasia or rosacea, or dandruff, in particular psoriasis or atopic dermatitis.

The extract or compositions of the invention may activate sirtuins, such as SIRT1. Thus, the present invention also provides an extract or composition of the invention for use as a sirtuin activator, in particular a SIRT1 activator. For example, the present invention provides an extract or composition of the invention for use as a sirtuin activator, in particular a SIRT1 activator, following an inflammatory stimulation.

Thus, the present invention also provides a plant extract or composition of the invention as a sirtuin activator for use in improving the barrier function of the skin and therefore the hydration of the skin, and/or an anti-ageing active agent, and/or an agent for the treatment of metabolic syndrome and associated diseases, and/or an agent for the treatment of degenerative diseases and/or associated disorders. Diseases/conditions include, but are not limited to, autoimmune diseases (such as systemic lupus erythematosus, rheumatoid arthritis, Gougerot-Sjögren's disease or Crohn's disease), cardiovascular diseases (such as heart disease), asthma, Alzheimer's disease, Huntington's disease, irritable bowel syndrome, cancers, type II diabetes, atopic dermatitis, osteoporosis, or COPD (chronic obstructive pulmonary disease).

There is also provided the use of an extract or composition of the invention in the manufacture of a medicament for treating or preventing inflammation, for activating PPARs and/or for activating sirtuins, such as SIRT1 as defined above.

There is also provided a method of treating of preventing inflammation, for activating PPARs and/or for activating sirtuins (such as SIRT1) as defined above, comprising the administration of a therapeutically effective amount of an extract or composition of the invention to a subject in need thereof.

Preventive and/or Treating Agent for Inflammation

As shown in the application, the plant extract according to the invention is suitable for the treatment of inflammation, and more particularly of chronic inflammation and all the associated disorders.

Accordingly, the plant extract according to the invention can be an anti-inflammatory, that is to say a drug for fighting inflammation, in particular for treating an inflammatory reaction and the conditions/diseases resulting therefrom.

The plant extract according to the invention may advantageously be an agent for the treatment of chronic inflammation, or of a chronic inflammatory disease.

More preferably, the plant extract according to the invention may be an agent for the treatment of chronic inflammation associated with skin disorders, namely, for example, atopic dermatitis, seborrhoeic dermatitis, acne, psoriasis, couperose, erythrosis, telangiectasia or rosacea, or dandruff.

As used herein “treatment or treating” includes:
reducing the severity of disease;
treating the symptoms of a disease;
preventive treatments, for preventing the occurrence of a disease, and
curative treatments, for curing a disease.

In particular, the term may refer to achieving a reduction in the severity of one or more clinical symptom associated with the disease or disorder (e.g. the fungal infection), as may be determined using techniques known to those skilled in the art (for example, by a medical physician) and/or to slowing the progression of the disease or disorder (i.e. increasing the amount of time taken for the disease or disorder to progress to a more severe state, e.g. when compared to the time expected to be taken in a patient not so treated).

As used herein, the term “prevention” (and, similarly, “preventing”) includes references to the prophylaxis of the disease or disorder (and vice-versa). In particular, the term may refer to achieving a reduction in the likelihood of the patient (or healthy subject) developing the condition (for example, at least a 10% reduction, such as at least a 20%, 30% or 40% reduction, e.g. at least a 50% reduction).

For the avoidance of doubt, in the context of the present invention, the terms “treating” and “preventing” include the therapeutic, or palliative, treatment of subjects/patients in need of, as well as the prophylactic treatment and/or diagnosis of patients which are susceptible to, the relevant disease states.

As used herein in relation to medical conditions, the term “reducing” may refer to making the observed quantity smaller or decrease in size.

As used herein, the terms “subject” and “patient” may be used interchangeably and include mammalian species (particularly humans).

The plant extract according to the invention may be an activator of the enzymatic systems necessary for the synthesis of the resolution molecules.

In particular, the plant extract according to the invention may advantageously have promoting activity in respect of the enzymatic systems of the family of the lipoxygenases (LOX), advantageously 5-LOX and/or 12-LOX and/or 15-LOX, which are key in the synthesis of lipid mediators leading to the resolution molecules.

The extract of the invention may increase levels of specialised pro-resolving mediators derived from arachidonic acid, eicosapentaenoic acid and/or docosahexaenoic acid, namely at least one of the following molecules:
derived from ARA: LTB4, 5-HETE, 12-HETE, 15-HETE;
derived from DHA: 14-HDoHE, 17-HDoHE, RvD1, RvD2, 7-Mar1, PD1, PDx;
derived from EPA: 18-HEPE.

The plant extract is likewise an agent that may be capable of stimulating the production of final resolution mediators, namely at least one of the following mediators: RVD1, RVD2, 7-Mar1 and PD1.

### Activating Agent of PPARs

The plant extract according to the invention may be used as an activating agent (agonist) of the peroxisome proliferator-activated receptors (PPARs), advantageously following an inflammatory stimulation.

Accordingly, the plant extract according to the invention may be an active ingredient having an ability to activate PPAR-related pathways within the organism, and thus to improve and to treat disorders associated with those metabolic pathways.

Peroxisome proliferator-activated receptors (also referred to as PPARs) are members of the superfamily of the nuclear receptors which regulate in particular the synthesis of lipids, glucose and amino acids within the organism.

The plant extract according to the invention may be an agonist of at least one of the PPARs, advantageously chosen from PPAR-α, β/σ, -γ.

In particular, the plant extract according to the invention may be suitable for potentiating the expression of the mRNAs of PPARaIpha and PPARbeta, in particular following an inflammatory stimulation.

Such a plant extract accordingly may have an indication in respect of pathologies associated with the PPAR-related pathways, in particular as:
an agent for the treatment of skin disorders and pathologies, such as psoriasis or atopic dermatitis, and/or
an agent for controlling oxidative stress at neuronal level, acting within the context of cerebral traumas, and/or
an agent for promoting the phenomenon of healing, and/or
an agent in the barrier function of the skin, by improving cellular cohesion, and/or
an agent for managing body weight and associated disorders.

More generally, such an extract may also be suitable for treating neurodegenerative diseases, pain, cancer, the immune system, skin disorders and the treatment of pathologies associated with overproduction of sebum; it is also intended to be used for protection against oxidative stress.

The PPARs are, therefore, involved in many biological processes.

Accordingly, the plant extract according to the invention may be of pharmacological interest in the treatment of metabolic disorders involving the PPARs and of the long-term consequences thereof, namely:

### a) Metabolic Syndrome

PPARα activators have proved that they are able to regulate obesity in rodents by increasing hepatic oxidation of fatty acids and by reducing the levels of circulating triglycerides, which are responsible for hypertrophy and hyperplasia of adipose cells. As regards the PPARγ ligands, they are involved in regulating the expression of leptin in adipose tissue. Leptin is a protein of which the main effect is to reduce the appetite.

### b) Immune Response

PPARγ has been recognised as playing a fundamental role in the immune response by its ability to inhibit the expression of cytokines and to direct the differentiation of immune cells towards anti-inflammatory phenotypes. Results of recent clinical trials suggest that the natural PPARγ agonists present in foods may be beneficial for human health.

### c) Healing/Barrier Function

The phenomenon of healing is a process which is divided into three phases: the inflammatory phase, the phase of keratinocyte migration, and the phase of re-epithelialisation (closure of the wound). PPARβ/σ is involved in the migration and adhesion of keratinocytes. There is a delicate balance between the first pro-inflammatory signals triggered by PPARβ/σ and the negative regulation pathways in the final stages of healing wounds. Fine adjustment of the process of healing is thus obtained, avoiding the formation of keloid scarring, for example, as well as any abnormal scarring. This latter point likewise uses the PPARγ pathway. Action on the PPARs therefore has a beneficial effect at barrier function level by acting on cellular adhesion and cohesion.

### d) Analgesic

The synthetic PPARα agonists act as broad-spectrum analgesics which act dose-dependently. It has been reported that the supraspinal administration of PPARα ligands such as perfluorooctanoic acid reduces peripheral oedema and/or inflammatory hyperalgesia. On the other hand, intrathecal administration of PPARγ ligands such as rosiglitazone reduces the clinical signs relating to neuropathic pain. This shows that these drugs, PPAR ligands, could be used as analgesic agents.

### e) Skin Disorders and Pathologies

Activation of the PPARα and γ pathways activates cell differentiation, regulates apoptosis and inhibits cell proliferation, thus having a positive effect in the context of skin pathologies such as psoriasis and atopic dermatitis which may or may not be of allergic origin. In fact, it has been described in patent EP1041977 that PPAR activators can act on skin disorders associated with an anomaly in epidermal cell differentiation. Therefore the following skin disorders may be mentioned by way of example: psoriasis, eczema, dermatitides, acne vulgaris, keratoses, including ichthyosis, and skin cancers. This anomaly in epidermal cell differentiation is generally accompanied by hyperproliferation of the epidermal cells.

In addition, it has been described in patent FR2004/003069 that PPAR activators are capable of inhibiting the production of sebum by regulating the size of the sebaceous glands. Excess sebum can serve as a support for the anarchic development of bacterial flora and cause comedones and/or acne lesions or, in the region of the scalp, abnormal desquamation associated with the presence of the yeast Malassezia, which is responsible for the production of dandruff. Disorders associated with the sebaceous function can likewise lead to dermatological disorders, in particular peri-oral dermatitides, pathologies associated with hyperplasia of the sebaceous glands such as hereditary hyperplasia of the sebaceous glands, or overproduction of sebum associated with hormonal disorders such as hyperandrogeny of endocrine origin.

### Agent for the Treatment of Skin Disorders

The plant extract according to the invention may be used as an agent for the treatment of skin disorders, in particular those associated with chronic inflammation.

The skin disorders in question include in particular psoriasis, eczema, lichen planus, skin lesions associated with lupus, dermatitides (such as atopic, seborrhoeic or solar dermatitides), keratoses (such as seborrhoeic, senile, actinic, photo-induced or follicular keratosis), acne vulgaris, keloids, nevi, warts, ichthyoses and skin cancers.

In this respect, the extract according to the invention may be capable of inducing expression of the mRNAs of structural or keratinisation proteins of the skin, namely, for example, LAMC2, FN1, ITGA2, IVL, KRT19, GBA, HAS2.

Furthermore, it is shown that the extract according to the invention may cause a synergistic effect on the expression of the mRNAs FN1, LAMC2, IVL and ITGA2 following an inflammatory stimulation.

### Activating Agent of Sirtuins

The plant extract according to the invention may be used as an activating agent of sirtuins, more particularly of SIRT1, advantageously following an inflammatory stimulation.

Accordingly, the plant extract according to the invention may be an active ingredient having the ability to activate SIRT1 pathways within the organism, and therefore more generally the ability to improve disorders associated with that metabolic pathway.

Accordingly, the plant extract according to the invention may be an active ingredient for applications as an agent for improving the barrier function of the skin, and therefore hydration of the skin, as an antioxidising agent, a DNA repair agent or an anti-ageing agent.

The plant extract according to the invention may also be an active ingredient for the prevention and treatment of degenerative diseases associated with ageing, cellular senescence, the harmful effects of oxidative stress, loss of integrity of the barrier function and improvement of pathologies associated therewith, such as atopic dermatitis.

Accordingly, the invention relates to a new activating agent of SIRT1 for improving and/or maintaining a healthy body composition, for improving and/or maintaining glucose or insulin management, maintaining good ageing, for improving and/or maintaining a healthy lipid level or the metabolism of fats, for preventing and/or treating excess weight, for improving and/or maintaining healthy energy homeostasis, for protecting cells, for repairing DNA and/or for maintaining physical strength and/or muscle mass during ageing.

In this respect, the composition or extract according to the invention may be able to slow the ageing process and to prevent chronic age-related diseases.

Accordingly, the invention relates to a new activating agent of SIRT1 for reducing the risk of developing obesity, which reduces the risk of developing type II diabetes, which reduces the risk of developing elevated blood lipid levels or which reduces the risk of developing atherosclerosis and/or cardiovascular diseases.

### Plant Extract as a Non-Therapeutic Active Agent

The invention also relates to the plant extract according to the invention as a non-therapeutic cosmetic or dermatological active ingredient.

This non-therapeutic use includes the treatment of the overproduction of sebum.

This non-therapeutic use also includes the use of the extract as a calming agent, as an anti-dandruff agent or as an anti-ageing agent.

The plant extract according to the invention can also be used in non-therapeutic applications, for example cosmetic applications, for example as:
an activating agent (agonist) of peroxisome proliferator-activated receptors (PPARs), and/or
an activating agent of sirtuins, more particularly of SIRT1, and/or
an agent for improving the barrier function of the skin, in particular following an inflammatory stimulation.

### BRIEF DESCRIPTION OF THE FIGURES

FIG. 1 depicts the increase in the concentration of lipid mediators after stimulation with a water decoction of Avera javanica or an extract of the invention.

The present invention will be further described by reference to the following, non-limiting Examples.

### EXAMPLE 1: METHOD FOR OBTAINING AN AERVA PLANT EXTRACT

An Aerva plant extract was obtained by carrying out the following method:

a) a plant raw material from Aerva javanica (Burnt) Juss. ex Schult. was ground to a particle size of from 100 μm to 50 mm;

b) the raw material was then contacted with 50% ethanol/50% water; dynamic extraction was carried out for 2 hours at 50° C.;

c) filtration was carried out before the plant material is again contacted with the extraction solvent;

d) the decolouring/deodorising step was carried out, as required, with the addition of 10% activated carbon, based on the dry extract present. Contacting was carried out for 30 minutes to one hour;
e) after filtration and concentration, the extract was dried on a solid support of the maltodextrin type or was formulated on a liquid support such as glycerol.

The amount of various compounds, including quercetin di-rhamnosyl glucoside, kaempferol coumaroyl rhamnosyf galactoside isomer, isorhamnetin rhamnosyl rutinoside, rutin, kaempferol coumaroyl rhamnosyl galactoside, and/or kaempferol di-coumaroyl rhamnosyl galactoside were determined using a classical HPLC method with UV or DAD detector for quantification and/or MS for identification and quantification of compounds.

UV monitoring was performed at 330 nm and the amount of target compounds were quantified by comparing peak area of the sample with peak area of reference compound of known concentration.

The results are shown in Table 1.

### EXAMPLE 2: STIMULATION OF THE LIPID RESOLUTION MEDIATORS ON A CELL MODEL EQUIPMENT AND METHOD

The action of the extract according to Example 1 was evaluated on the synthesis of 7 lipids positioned at the strategic intersections of the metabolism of the PUFAs (polyunsaturated fatty acids: arachidonic acid (ARA), eicosapentaenoic acid (EPA), docosahexaenoic acid (DHA)), ARA (5-HETE, 12-HETE, 15-HETE), DHA (14-HDoHE, 17-HDoHE), EPA (18-EPE).

The evaluation of these 7 lipids thus made it possible to establish whether the enzymatic paths involved in the synthesis of the resolution mediators, which play a crucial role in the context of chronic inflammation, have or have not been mobilised by the extract according to Example 1. The final metabolites derived from the PUFAs were thus evaluated in the same cell model.

For all the experiments conducted, only the non-cytotoxic doses were evaluated.

After testing from 0.0064 to 500 μg/ml, the non-cytotoxic doses of 11, 33, 100 and 200 μg/ml were retained for the remainder of the work. The cytotoxicity test was carried out on normal human epidermal keratinocytes (NHEK, Promocell), and the test on the synthesis of the resolution molecules was carried out on a co-culture of dendritic cells (DC) and NHEK.

In order to carry out the cytotoxicity test, the NHEKs were cultivated in the presence of the active agents at doses ranging from 0.0064 to 500 μg/ml for 24 hours in culture medium at a density of 250 cells/cm² in a 96-well dish. During the final 6 hours, Alamar Blue (Invitrogen: DAL1025) was introduced into the medium. This reagent contains resazurin, a non-fluorescent indicator which is reduced to resorufin, a fluorescent indicator, by metabolically active cells. The level of fluorescence produced is therefore proportional to the number of living cells. Excitation is carried out at 530 nm and reading is carried out at 590 nm. At the end of this experiment, a non-cytotoxic concentration for each active agent was selected for evaluation on the synthesis of the lipid mediators.

In order to carry out the test of demonstrating the activation of resolution pathways, primary human monocytes were seeded and differentiated by means of GM-CSF (10 ng/ml) and IL-4 (10 ng/ml); in parallel, keratinocyte primary culture cells (NHEK) were prepared. After culture for one week, the NHEKs were deposited in inserts and cultured without contact with the cells which have become dendritic (DC). The DC/NHEK co-culture system was pre-incubated in the presence of the active agent at a concentration of 200 μg/ml for 4 hours. The inflammatory response was then triggered by the association of phorbol myristate (PMA—0.05 μM) and calcium ionophore (A23187—1 μM), in the presence of a mixture of eicosapentaenoic acid (EPA—1 μg/ml) and docosahexaenoic acid (DHA—1 μg/ml) (ratio 1/1). The addition of EPA and DHA was necessary to lower the sensitivity threshold and thus allow the resolution molecules to be measured. The supernatants were recovered after 2 hours and 4 hours for analysis by mass spectrometry and for quantification of the molecules derived from arachidonic acid (5-HETE, 12-HETE, 15-RETE), from DHA (14-HDoHE, 17-HDoHE), and from EPA (18-HEPE).

Experiments were carried out in triplicate for each experimental condition.

In order to assay the lipid mediators, the supernatants were defrosted on ice and the lipid compounds were concentrated by solid phase extraction (SPE), taken up in methanol before analysis. The analytical method used consists of separating the different analytes by high pressure liquid chromatography as a function of their retention time and quantifying them by mass spectrometry. The analyses were carried out on a LC 1290 Infinity chain (Agilent Technologies) coupled to a 6460 Triple Quad LC/MS mass spectrometer (Agilent Technologies) equipped with an electrospray ionisation source (Jet stream technology) operating in negative mode. The chromatographic separations were carried out on a ZorBAX SB-C18 column.

The doses of each marker were then converted by calculation in order to obtain the percentage activation relative to the control stimulated with PUFA of the plate using the following calculation:

% activation=100×(value of the active agent−value of the PUFA control)/value of the PUFA control

### Results of the Evaluation of the Biomarkers by Mass Spectrometry

At the end of the inflammatory stimulation time, the supernatants were recovered for analysis by mass spectrometry and for quantification of the molecules derived:
from arachidonic acid: LTB4, 5-HETE, 12-HETE, 15-HETE;
from DHA: 14-HDoHE, 17-HDoHE, RvD1, RvD2, 7-Mar1, PD1, PDx;
from EPA: 18-HEPE.

These results are summarised in Tables 2 and 3 below:

TABLE 2
Dose of the lipid mediators of the resolution pathways after stimulation (2 h and 4 h) by the Aerva extract at 200 μg/ml

| | 18- | 15- | 17- | 14- | 12- | 5- |
|---|---|---|---|---|---|---|
| | HEPE | HETE | HDOHE | HDOHE | HETE | HETE |
| AVERAGE pg/well | | | | | | |
| 2 h CONTROL PUFA + PMA/A23187 | 3373 | 0 | 1094 | 285 | 515 | 27 |
| 2 h Aerva 200 μg/ml + PUFA + PMA/A23187 | 3805 | 47 | 2013 | 607 | 780 | 114 |
| 4 h CONTROL PUFA + PMA/A23187 | 2256 | 113 | 709 | 77 | 160 | 8 |
| 4 h Aerva 200 μg/ml + PUFA + PMA/A23187 | 3311 | 90 | 1572 | 296 | 459 | 39 |
| SEM | | | | | | |
| 2 h CONTROL PUFA + PMA/A23187 | 74 | 0 | 118 | 45 | 73 | 21 |
| 2 h Aerva 200 μg/ml + PUFA + PMA/A23187 | 81 | 8 | 106 | 30 | 25 | 16 |
| 4 h CONTROL PUFA + PMA/A23187 | 57 | 29 | 21 | 6 | 17 | 5 |
| 4 h Aerva 200 μg/ml + PUFA + PMA/A23187 | 113 | 11 | 110 | 22 | 25 | 10 |
| % activation relative to the control | | | | | | |
| 2 h CONTROL PUFA + PMA/A23187 | 0 | 0 | 0 | 0 | 0 | 0 |
| 2 h Aerva 200 μg/ml + PUFA + PMA/A23187 | 13 | 46,772 | 84 | 113 | 52 | 318 |
| 4 h CONTROL PUFA + PMA/A23187 | 0 | 0 | 0 | 0 | 0 | 0 |
| 4 h Aerva 200 μg/ml + PUFA + PMA/A23187 | 47 | −21 | 122 | 283 | 187 | 385 |
| p t-test | | | | | | |
| 2 h CONTROL PUFA + PMA/A23187 | 1.00000 | 1.00000 | 1.00000 | 1.00000 | 1.00000 | 1.00000 |
| 2 h Aerva 200 μg/ml + PUFA + PMA/A23187 | | | | | | |
| 4 h CONTROL PUFA + PMA/A23187 | 1.00000 | 1.00000 | 1.00000 | 1.00000 | 1.00000 | 1.00000 |
| 4 h Aerva 200 μg/ml + PUFA + PMA/A23187 | | 0.50036 | | | | 0.05123 |

TABLE 3
Dose of the resolution molecules after stimulation by the Aerva extract at 200 μg/ml

| | RVD2 | RVD1 | 7(S)MaR1 | PDX | PD1 | LTB4 |
|---|---|---|---|---|---|---|
| AVERAGE pg/well | | | | | | |
| 2 h CONTROL PUFA + PMA/A23187 | 0 | 2 | 18 | 0 | 3 | 14 |
| 2 h Aerva 200 μg/ml + PUFA + PMA/A23187 | 11 | 4 | 22 | 1 | 12 | 75 |
| 4 h CONTROL PUFA + PMA/A23187 | 0 | 0 | 0 | 0 | 0 | 26 |
| 4 h Aerva 200 μg/ml + PUFA + PMA/A23187 | 0 | 2 | 19 | 0 | 7 | 54 |
| SEM | | | | | | |
| 2 h CONTROL PUFA + PMA/A23187 | 0 | 1 | 6 | 0 | 1 | 3 |
| 2 h Aerva 200 μg/ml + PUFA + PMA/A23187 | 6 | 2 | 3 | 0 | 1 | 13 |
| 4 h CONTROL PUFA + PMA/A23187 | 0 | 0 | 0 | 0 | 0 | 5 |
| 4 h Aerva 200 μg/ml + PUFA + PMA/A23187 | 0 | 2 | 5 | 0 | 1 | 5 |
| % activation relative to the control | | | | | | |
| 2 h CONTROL PUFA + PMA/A23187 | 0 | 0 | 0 | 0 | 0 | 0 |
| 2 h Aerva 200 μg/ml + PUFA + PMA/A23187 | 10,699 | 74 | 24 | 81 | 264 | 439 |
| 4 h CONTROL PUFA + PMA/A23187 | 0 | 0 | 0 | 0 | 0 | 0 |
| 4 h Aerva 200 μg/ml + PUFA + PMA/A23187 | 0 | 505 | 18,963 | 284 | 1,376 | 104 |
| p t-test | | | | | | |
| 2 h CONTROL PUFA + PMA/A23187 | 1.00000 | 1.00000 | 1.00000 | 1.00000 | 1.00000 | 1.00000 |
| 2 h Aerva 200 μg/ml + PUFA + PMA/A23187 | 0.13805 | 0.52707 | 0.53535 | 0.63708 | | |
| 4 h CONTROL PUFA + PMA/A23187 | 1.00000 | 1.00000 | 1.00000 | 1.00000 | 1.00000 | 1.00000 |
| 4 h Aerva 200 μg/ml + PUFA + PMA/A23187 | 1.00000 | 0.45644 | | 0.37390 | | |

It is observed that PMA/A23187 stimulation, in the presence of the PUFAs, activates the enzymatic systems necessary for the synthesis of the resolution molecules.

An increase in 5-HETE, 12-HETE and 15-HETE, which are indicators of 5-lipoxygenase, 12-lipoxygenase and 15-lipoxygenase enzymatic activities, respectively, is observed.

Finally, secretion of 14-HDOHE, 17-HDOHE and 18-HEPE, which are precursors of the resolution molecules, is also observed. These results are in accordance with what is expected.

An increase in the secretion of the synthesis intermediates involved in the resolution of inflammation is observed under the effect of the extract according to Example 1. This increase is significant for all the intermediates, the percentage of activation tending to increase (except for 15-HETE) during the stimulation time.

Regarding the final resolution mediators, a tendency to increase the secretion of RVD1 and RVD2 was observed. A significant increase in the secretion of two other metabolites: 7(s)maresin and PD1, is also observed.

These elements therefore support a pro-resolvent role of the Aerva extract.

### EXAMPLE 3: ACTIVATION OF THE RESOLUTION PATHWAYS AND RECEPTORS ON A RECONSTRUCTED SKIN MODEL

Reconstructed human epidermis (RHE) modules were used in the experiments, and the experiments were carried out in triplicate (three wells).

The modules were supplied by Skinethic in the 12-day development stage and exhibit a keratinised surface in contact with the air and then a deep surface of non-keratinised cells in contact with the culture medium.

They were maintained in culture for a further 4 days before being stimulated by application of the active agent topically and in the culture medium.

Topical stimulation was possible by diluting the active agent in the culture medium and applying the solution to the surface of the RHE module for 24 hours.

At the end of these 24 hours, the medium in contact with the keratinised cells was aspirated carefully in order to permit air-liquid culture again. The growth and stimulation medium is a medium supplied directly by the manufacturer, specific to the maintenance of RHEs.

In order to test the viability of the RHEs with regard to the extract according to Example 1, the RHEs were incubated in the presence of the active agent applied topically and in the culture medium for 24 hours as described previously.

At the end of these 24 hours, the stimulation applied topically was withdrawn in order to permit air-liquid culture for 24, 48 or 72 hours in the presence of Alamar blue, with a reading of the fluorescence at each of those times. Following this test, the test concentration of 500 μg/ml was selected for the remainder of the study.

The RHE modules were incubated in the presence of the active agent at the two doses selected during the viability test. The active agent was applied for 24 hours topically and in the cell culture medium as described previously. At the end of these 24 hours, the stimulation applied topically was withdrawn in order to permit air-liquid culture for a further 24 hours.

An inflammatory stimulation (Mix CTK), corresponding to a cocktail of OSM, TNF-a and IL-17 each at 10 ng/ml (chosen according to the publication of Boniface K et al. J. Immunol. 2007, 178: 4615), was then added or not added during the 24 hours of air-liquid culture in the medium below the RHEs. The solvent of the active agent was introduced into the control wells.

At the end of the stimulation step, the cells were separated from their support by means of dispase and were then recovered and lysed by means of a precellys in the presence of qiazol. After phenol/chloroform extraction and passage over a cleansing column, the RNAs were taken up in milliQ water before being assayed on nanodrop and frozen for subsequent use. Reverse transcription was then performed by means of the Maxima First stand cDNA synthesis kit (ThermoFisher), and then the specific steps of preparation for the chip (96×96) according to the Fluidigm protocol version PN100-1201B1 were carried out.

The level of expression of the mRNAs was normalised with the following reference genes: YWHAZ, GAPDH, HPRT1 and ACTB.

The results are expressed as the number of induction times in relation to the DMSO control at 10⁻⁵ M using the Livak method commonly called DDCt (Delta Delta Ct). The Cts correspond to the number of cycles necessary to generate a fluorescent signal above the predefined threshold. For each gene, the values are expressed relative to the control, which is equal to 1.

Under inflammatory stimulation, expression of the following genes was monitored (Table 4):

TABLE 4

| | | Inflammatory | Aerva | | p value |
|---|---|---|---|---|---|
| Gene | Control | stimulation | extract | Ratio | (t test) |
| LTA4H | 1.00 | 1.72 | 3.02 | 1.75 | 0.04028 |
| cPLA2-IVA | 1.00 | 0.23 | 0.27 | 1.21 | 0.25203 |
| eLOX3 | 1.00 | 0.91 | 1.66 | 1.83 | 0.27019 |
| 15LOX1 | 1.00 | 0.81 | 1.17 | 1.44 | 0.46207 |
| 12(S)LOX | 1.00 | 0.35 | 0.03 | 0.10 | 0.46607 |

In the presence of the inflammatory cocktail, it was possible to show that the Aerva extract induces an increase in the expression of the LTA4 hydrolase gene (LTA4H p=0.04028), an enzyme involved in the synthesis of LTB4.

These results are perfectly consistent with the experiments conducted on the DC/NHEK model, where it was observed that the active agent, under inflammatory conditions, increased the expression of LTB4.

In addition, a tendency to increase the mRNAs of eLOX-3, an enzyme which is potentially involved in the synthesis of the resolution molecules owing to the 5-lipoxygenase activity thereof, has also been noted. Likewise, the expression of 15LOX1 is very slightly increased. These results are also consistent with those obtained on the DC/NHEK model, where an increase in 15-HETE and 5-HETE, which are indicators of 15-lipoxygenase and 5-lipoxygenase activities, respectively, was observed. 15-LOX enzymatic activity in particular is necessary for the synthesis of protectin D1, thus consolidating the synthesis observed in Example 2 of PD1 under the effect of the Aerva extract.

In view of the results obtained on the keratinocyte-dendritic cell model, these inductions permit the establishment of a positive control loop for inflammation owing to the induction of the precursors of the resolution molecules, which are dependent on these enzymes. These results are wholly in line with the ability of the Aerva extract to act at the level of the control and of the resolution of a chronic inflammation.

In addition, the genes associated with the resolution receptors were monitored after stimulation of the RHE by the Aerva extract (Table 5).

TABLE 5

| | | Aerva | p value |
|---|---|---|---|
| Gene | Control | extract | (t-test) |
| CMKLR1 | 1.00 | 2.43 | 0.36377 |
| GPR32 | 1.00 | 2.64 | 0.36421 |
| FPR2 | 1.00 | 1.62 | 0.58771 |

The expression of the receptors directly associated with resolution was activated by the Aerva extract such as the resolvin E1 receptor CMKRL1 and GPR32, the resolvin D1 receptor and FPR2, the lipoxin A4 receptor.

After inflammatory stimulation, an identical monitoring was carried out (Table 6):

TABLE 6

| | | | Aerva | | |
|---|---|---|---|---|---|
| | | | extract + | | |
| | | Inflammatory | inflammatory | | p value |
| Gene | Control | stimulation | stimulation | Ratio | (t-test) |
| FPR2 | 1.00 | 5.43 | 17.90 | 3.30 | 0.01238 |
| GPR32 | 1.00 | 8.08 | 23.03 | 2.85 | 0.05332 |
| CMKLR1 | 1.00 | 7.20 | 20.64 | 2.87 | 0.07744 |

In the presence of the inflammatory stimulation represented by the mix of cytokines, it was possible to measure that the Aerva extract potentiates the expression of the mRNAs of the receptors described in the preceding paragraph, with in particular the significant induction of FPR2 (p=0.01238), the lipoxin A4 receptor, CMKRL1 (p=0.07858), the resolvin E1 receptor, and GPR32 (p=0.05332), the resolvin D1 receptor.

### EXAMPLE 4: ACTIVATION OF THE SKIN PROTECTION PATHWAYS ON A RECONSTRUCTED SKIN MODEL

As described in Example 3, the Aerva extract was contacted at a concentration of 500 μg/ml in a reconstructed skin model.

After incubation for 24 hours, the following genes were stimulated (Table 7):

TABLE 7

| | | Aerva | |
|---|---|---|---|
| Gene | Control | extract | p (t-test) |
| IL1B | 1.00 | 10.92 | 0.00504 |
| IL1A | 1.00 | 5.61 | 0.00685 |
| RNASE7 | 1.00 | 2.85 | 0.01374 |
| IL17A | 1.00 | 2.21 | 0.01932 |
| IL12A | 1.00 | 2.52 | 0.01941 |

The Aerva extract induces the skin protection mechanisms since it was possible to measure a significant increase in the mRNAs of IL1 B (p=0.00504), of ILIA (p=0.00685), of IL17A (p=0.01932) and of IL12A (p=0.01941), which are involved directly in the mobilisation of the skin defence systems, and an induction of the mRNAs associated with antimicrobial peptides (RNASE7 (p=0.01374)).

### EXAMPLE 5: ACTIVATION OF THE INFLAMMATION CONTROL PATHWAYS ON A RECONSTRUCTED SKIN MODEL

As described in Example 3, the Aerva extract was contacted at a concentration of 500 μg/ml in a reconstructed skin model in the presence of a pro-inflammatory stimulation.

After incubation for 24 hours, the following genes were stimulated (Table 8):

TABLE 8

| | | | Aerva | | |
|---|---|---|---|---|---|
| | | | extract + | | |
| | | Inflammatory | inflammatory | | |
| Gene | Control | stimulation | stimulation | Ratio | p value |
| PI3 | 1.00 | 8.04 | 17.43 | 2.17 | 0.00489 |
| IL17A | 1.00 | 0.96 | 2.17 | 2.26 | 0.02028 |
| IL23 | 1.00 | 1372.82 | 357.56 | 0.26 | 0.08658 |
| DEFB4 | 1.00 | 53634.96 | 4988.77 | 0.09 | 0.14170 |
| IL6 | 1.00 | 530.80 | 18.83 | 0.04 | 0.19263 |

It was found that the Aerva extract has a tendency to reverse the effect of the inflammatory stimulation to a greater extent than it succeeds in inducing the potentiation thereof.

Accordingly, it has valuable effects of controlling the inflammatory reaction without paralysing it totally, thus permitting the expression of important activities such as the defence activities (DEFB4 p=0.1470). These results are wholly in keeping with an action of controlling a chronic inflammatory state, which tends to be persistent and latent.

### EXAMPLE 6: ACTIVATION OF THE PATHWAYS OF CONTROL OF THE GENERAL METABOLISM AND OF DEFENCES AGAINST OXIDATIVE STRESS—PPAR ACTIVATION

As described in Example 3, the Aerva extract was contacted at a concentration of 500 μg/ml in a reconstructed skin model.

After incubation for 24 hours, the following genes were stimulated (Table 9):

TABLE 9

| | | Aerva | p value |
|---|---|---|---|
| Gene | Control | extract | (t-test) |
| PPARbeta | 1.00 | 3.23 | 0.00006 |
| PPARalpha | 1.00 | 1.52 | 0.02723 |
| PPARgamma | 1.00 | 1.65 | 0.12356 |

The Aerva extract induces an increase in the expression of the mRNAs of the transcription factors PPARbeta (p=0.00006), PPARalpha (p=0.02723) and PPARgamma (p=0.12356), which are involved in broad physiological controls such as fatty acid metabolism, the control of inflammation or the phenomena of healing, for example.

Activation of these pathways clearly indicates that the Aerva extract plays a controlling role both at the level of a chronic inflammatory phenomenon but also for stimulating a response of the organism towards oxidative stress.

The PPARs play a role in the general homeostasis of the organism and are therapeutic targets in various domains, with an action in skin disorders, and in particular pathologies of the order of chronic inflammation (atopic dermatitis, psoriasis, for example) as well as healing, management of body weight, neurodegenerative diseases or cancer (Sertznig et al., 2008; Yessoufou et al., 2010).

After inflammatory stimulation, the same genes were monitored (Table 10):

TABLE 10

| | | | Aerva | | |
|---|---|---|---|---|---|
| | | | extract + | | |
| | | Inflammatory | inflammatory | | p value |
| Gene | Control | stimulation | stimulation | Ratio | (t-test) |
| PPARbeta | 1.00 | 3.55 | 7.10 | 2.00 | 0.01845 |
| PPARalpha | 1.00 | 0.90 | 1.85 | 2.05 | 0.04015 |

In the presence of the inflammatory stimulation, it was possible to show that the Aerva extract potentiates the expression of the mRNAs of PPARbeta (p=0.01845) and alpha (p=0.04015).

### EXAMPLE 7: ACTIVATION OF THE DERMIS-EPIDERMIS CELLULAR COHESION PATHWAYS, PLAYING A ROLE IN THE BARRIER FUNCTION

As described in Example 3, the Aerva extract was contacted at a concentration of 500 μg/ml in a reconstructed skin model.

After incubation for 24 hours, the following genes were stimulated (Table 11):

TABLE 11

| | | Aerva | p value |
|---|---|---|---|
| Gene | Control | extract | (t-test) |
| GBA | 1.00 | 1.91 | 0.00075 |
| LAMC2 | 1.00 | 2.41 | 0.01523 |
| FN1 | 1.00 | 3.53 | 0.01569 |
| ITGA2 | 1.00 | 1.21 | 0.04782 |
| IVL | 1.00 | 2.06 | 0.06220 |
| HAS2 | 1.00 | 3.29 | 0.11201 |
| KRT19 | 1.00 | 4.66 | 0.22774 |

It was possible to demonstrate that the Aerva extract induces the expression of the mRNAs of structural or keratinisation proteins of the skin, such as laminin LAMC2 (p=0.01523), which constitutes the basal membrane, fibronectin FN1 (p=0.01569), permitting the junction between the epidermis and the dermis, integrin ITGA2 (p=0.04782), which is involved in the binding of cells to the extracellular matrix, involucrin IVL (p=0.06220), which is involved in protection and the stratum corneum, cytokeratin KRT19 (p=0.22774), and also a significant overexpression of the mRNAs of GBA (p=0.00075), which is involved in the barrier function of the skin. It has also been shown that the mRNAs of the enzyme involved in the synthesis of hyaluronic acid HAS2 (p=0.11201) were induced.

These modifications indicate an effect of the active agent on the remodelling of the dermal matrix and the improvement of the barrier function, which is important in skin disorders inducing a loss of skin hydration. A loss of the barrier function will in fact cause an increase in imperceptible water loss.

After inflammatory stimulation, the following genes were monitored (Table 12):

TABLE 12

| | | | Aerva | | |
|---|---|---|---|---|---|
| | | | extract + | | |
| | | Inflammatory | inflammatory | | p value |
| Gene | Control | stimulation | stimulation | Ratio | (t-test) |
| FN1 | 1.00 | 11.61 | 29.13 | 2.51 | 0.01335 |
| LAMC2 | 1.00 | 2.75 | 5.19 | 1.89 | 0.04636 |
| IVL | 1.00 | 0.89 | 1.98 | 2.24 | 0.05376 |
| ITGA2 | 1.00 | 1.23 | 1.88 | 1.52 | 0.05692 |
| EGF | 1.00 | 0.13 | 0.36 | 2.83 | 0.17512 |
| HAS2 | 1.00 | 5.30 | 1.20 | 0.23 | 0.21999 |
| KRT6A | 1.00 | 1.09 | 1.52 | 1.40 | 0.27101 |
| COL1A1 | 1.00 | 0.28 | 0.63 | 2.21 | 0.28914 |
| KRT19 | 1.00 | 9.29 | 13.47 | 1.45 | 0.62749 |

After inflammatory stimulation, the Aerva extract induces a synergy of effect on the expression of the mRNAs of FN1, LAMC2, IVL, ITGA2 by increasing them relative to the inflammatory stimulation on its own.

The expression of the mRNAs of the epithelial growth factor EGF as well as of COL1A1 was inhibited in the presence of the inflammatory stimulation. The addition of the Aerva extract will regulate this reduction by restoring in part the expression at its basal level.

### EXAMPLE 8: ACTIVATION OF THE PATHWAYS OF PROTECTION AGAINST OXIDATIVE STRESS AND SKIN AGEING

As described in Example 3, the Aerva extract was contacted at a concentration of 500 μg/ml in a reconstructed skin model.

After incubation for 24 hours, the following genes were stimulated (Table 13):

TABLE 13

| | | Aerva | p value |
|---|---|---|---|
| Gene | Control | extract | (t-test) |
| HMOX1 | 1.00 | 1.85 | 0.00417 |
| HBEGF | 1.00 | 4.58 | 0.01701 |
| SIRT1 | 1.00 | 2.66 | 0.02794 |

The Aerva extract induced a significant overexpression of heme oxygenase HMOX1 (p=0.00417), of HBEGF (p=0.01701) and of SIRT1 (p=0.02794), all of which are involved in protecting the skin by increasing the anti-oxidative defences of the skin and limiting cellular senescence.

After inflammatory stimulation, the following genes were monitored (Table 14):

TABLE 14

| | | | Aerva | | |
|---|---|---|---|---|---|
| | | | extract + | | |
| | | Inflammatory | inflammatory | | p value |
| Gene | Control | stimulation | stimulation | Ratio | (t-test) |
| SIRT1 | 1.00 | 2.62 | 5.97 | 2.28 | 0.04739 |
| SFN | 1.00 | 1.03 | 1.72 | 1.68 | 0.14968 |
| HMOX1 | 1.00 | 1.32 | 2.14 | 1.63 | 0.18469 |

After inflammatory stimulation, it is observed that the active agent has had a significant synergistic effect on the increase in the expression of SIRT1 (p=0.04739) and a tendency to increase HMOX1 and SFN.

### EXAMPLE 9: COMPARISON OF LIPID MEDIATOR SYNTHESIS AFTER STIMULATION WITH EITHER A WATER DECOCTION OF AVERA JAVANIC OR AN EXTRACT OF THE INVENTION

The extract of the invention was prepared as described in Example 1.

The decoction was prepared by contacting plant raw material from Aerva javanica (Burm.f.) Juss. Ex Schult with water at 50° C. for 2h prior to filtering and drying using the techniques previously described in Example 1.

Stimulation was carried out using the method described in Example 2.

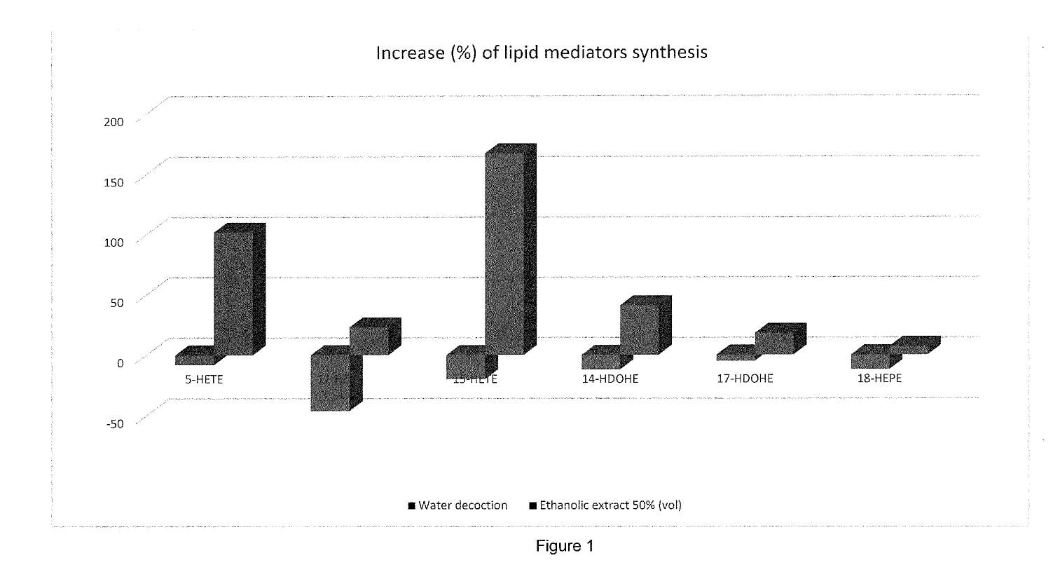

The results after stimulation are shown in FIG. 1.

1. A plant extract obtained from or obtainable from the aboveground part of a plant of the genus Aerva.

2. A plant extract according to claim 1, wherein the plant of the genus Aerva is Aerva javanica (Burm.f.) Juss. ex Schult.

3. A plant extract according to claim 1, wherein the extract comprises from about 0.1 to about 0.3% quercetin di-rhamnosyl glucoside, from about 0.05 to about 0.1% kaempferol coumaroyl rhamnosyl galactoside isomer, from about 0.04 to about 0.08% isorhamnetin rhamnosyl rutinoside, from about 0.5 to about 0.15% rutin, from about 0.05 to about 0.1% kaempferol coumaroyl rhamnosyl galactoside, and from about 0.1 to about 0.3% kaempferol di-coumaroyl rhamnosyl galactoside.

4. A plant extract according to claim 1, characterised in that the plant extract is obtained by or obtainable by an extraction method comprising a step of contacting the aboveground part of a plant of the genus Aerva with at least one physiologically acceptable extraction solvent, optionally followed by a step of removing the solvent.

5. A plant extract according to claim 4, characterised in that the extraction solvent is chosen from polar solvents, eutectic solvents, supercritical CO₂ or mixtures thereof.

6. A plant extract according to claim 4, characterised in that the extraction method comprises, following the contacting step, a decolouring step and/or a deodorising step.

7. A plant extract according to claim 4, wherein the extraction solvent is an alcoholic solvent or an aqueous-alcoholic solvent.

8. (canceled)

9. A composition comprising:
(i) a plant extract according to claim 1, and
(ii) at least one physiologically acceptable excipient.

10. A composition according to claim 9, wherein the composition is a cosmetic, pharmaceutical or food composition.

11. A composition according to claim 9, wherein the physiologically acceptable excipient is a dermatologically acceptable excipient.

12-35. (canceled)

36. A method of preventing and/or treating inflammation comprising the administration of a plant extract according to claim 1, or a composition comprising said extract and at least one physiologically acceptable excipient, to a subject in need thereof.

37. A method according to claim 36, wherein the inflammation is chronic inflammation.

38. A method according to claim 37, wherein the chronic inflammation is associated with a skin disorder.

39. A method of activating peroxisome proliferator-activated receptors (PPARs) comprising the administration of a plant extract according to claim 1, or a composition comprising said extract and at least one physiologically acceptable excipient, to a subject in need thereof.

40. A method according to claim 39, wherein the activation of PPARs is following an inflammatory stimulation.

41. A method according to claim 39 for treating skin disorders and pathologies, and/or controlling oxidative stress at neuronal level, acting within the scope of cerebral traumas, and/or promoting the phenomenon of healing, and/or improving cellular cohesion, and/or managing body weight and/or associated disorders.

42. A method according to claim 41, wherein the skin disorders are associated with chronic inflammation.

43. A method according to claim 42, wherein the skin disorders and pathologies are selected from psoriasis or atopic dermatitis.

44. A method of activating sirtuins comprising the administration of a plant extract according to claim 1, or a composition comprising said extract and at least one physiologically acceptable excipient, to a patient in need thereof.

45. A method according to claim 44, wherein the sirtuin is SIRT1.

46. A method according to claim 44, wherein the activation of sirtuins or SIRT1 is following an inflammatory stimulation.

47. A method according to claim 44 for improving the barrier function of the skin and therefore the hydration of the skin, and/or as an anti-ageing active agent, and/or for treating metabolic syndrome and associated diseases, and/or for treating degenerative diseases and/or associated disorders.

48. (canceled)

49. A method for producing a plant extract according to claim 1, comprising a step of contacting the aboveground plant part of the genus Aerva with at least one physiologically acceptable extraction solvent, optionally followed by a step of removing at least part of the solvent.

50. A method according to claim 49, characterised in that the extraction solvent is chosen from polar solvents, eutectic solvents, supercritical CO₂ or mixtures thereof.

51. A method according to claim 49 characterised in that the extraction method comprises, following the contacting step, a decolouring step and/or a deodorising step.

52. A method for producing a plant extract, according to claim 49,
comprising the following successive steps:
a) a step of preparing/providing the aboveground part of the plant of the genus Aerva,
b) a step of contacting said aboveground part with the extraction solvent,
c) a step of filtering the mixture to obtain a filtrate of interest, optionally subjecting the solid residue of the aboveground part to a further contacting step followed by a filtering step,
d) optionally, a step of decolouring and/or deodorising said filtrate of interest, and
e) optionally, a step of formulating said plant extract, on a solid support or on a liquid support.

53. A method according to claim 52, further including a concentration step after step c) and/or after step d).

54. A method according to claim 49, wherein the extraction solvent is an alcoholic solvent or an aqueous-alcoholic solvent.
